(19) United States
(12) Patent Application Publication
(10) Pub. No.: US 2025/0163384 A1
(43) Pub. Date: May 22, 2025

(54) ORGANOIDS DERIVED FROM HUMAN COLONIC CELLS AND METHODS OF MAKING AND USING THE SAME

(71) Applicant: ARIZONA BOARD OF REGENTS ON BEHALF OF THE UNIVERSITY OF ARIZONA, Tucson, AZ (US)

(72) Inventors: Kelvin W. POND, Tucson, AZ (US); Curtis THORNE, Tucson, AZ (US); Andrew PAEK, Tucson, AZ (US)

(21) Appl. No.: 18/729,803

(22) PCT Filed: Feb. 4, 2023
(86) PCT No.: PCT/US2023/012351

§ 371 Date: Jul. 17, 2024

Related U.S. Application Data
(60) Provisional application No. 63/267,551, filed on Feb. 4, 2022.

(51) Int. Cl. C12N 5/071; C12M 1/32; C12M 1/34

(52) U.S. Cl. CPC C12N 5/0679; C12M 23/12; C12M 41/38; C12N 2513/00

(57) ABSTRACT

A human 2D patient-derived organoid screening platform to study tissue patterning and kinase pathway dynamics in single cells. With the help of this system, it is discovered that waves of ERK signaling induced by apoptotic cells play a critical role in maintaining tissue patterning and homeostasis.

### RELATED APPLICATION

This application claims priority to U.S. Provisional Application 63/267,551 filed on Feb. 4, 2022, the content of which is incorporated herein by reference in its entireties for all purposes.

### GOVERNMENT RIGHTS

This invention was made with government support under Grant Nos. DK103126, GM130864, and GM147128 awarded by National Institutes of Health. The government has certain rights in the invention.

### BACKGROUND

Establishment and maintenance of different cellular compartments in tissues are universal requirements across all metazoans. Maintaining the correct ratio of cell types in time and space allows tissues to form patterned compartments and perform complex functions. Patterning is especially evident in the human colon, where tissue homeostasis is maintained by stem cells in crypt structures that balance proliferation and differentiation.

An in vitro cellular system that mimics the compartmentalization and patterning of the human colon would be useful for studying the structure and function of the colon. However, it is difficult to replicate the cellular patterning and spacing in vitro. Although a murine line has been established, it was derived from primary cells and is difficult and expensive to maintain.

### SUMMARY

The present disclosure provides methods of generating a unique organoid cell line derived from human colon adenomatous polyps. The disclosed human colon organoid demonstrates patterning in vitro that is similar to those observed in vivo, and is amenable to live tracking of single cell, and single cells analysis of kinase signaling dynamics, cellular movement, traction force in the organoid, and analysis of electric cell-substrate impedance sensing, among others.

In one embodiment, an organoid comprising a plurality of cells derived from a human is disclosed, wherein the organoid comprises at least two compartments: a first compartment and a second compartment. The first compartment comprises at least 10%, or 40%, or 50%, or 60%, or 80%, or 90%, or even 99% undifferentiated stem cells, and the second compartment comprises at least 60%, or 70%, or 80%, or 90% differentiated cells and less than 0.1%, or less than 0.5%, or less than 1% undifferentiated stem cells.

In one aspect, the plurality of cells in the disclosed organoid is derived from normal colon cells, or from adenomatous polyp. In another aspect, the plurality of cells in the disclosed organoid is of tumor origin and therefore lacks contact inhibition.

In another embodiment, the plurality of cells in the disclosed organoid forms a monolayer on a tissue culture plate.

In some other embodiments, in the disclosed organoid, Erk kinase activity is lower in the first compartment than Erk kinase activity in the second compartment. In one aspect, the Erk kinase activity forms a gradient that increases from the first compartment to the second compartment.

In some other embodiments, a method for preparing an organoid is disclosed, which comprises these steps: a) digesting a biopsy sample obtained from human intestine with collagenase to form a single cell suspension, b) allowing cells of the single cell suspension to grow, c) transferring single cells in the suspension from step (b) onto one or more wells of a multi-well plate, said one or more wells being coated with extracellular matrix, and d) allowing single cells to grow and self-organize to form a 2D organoid monolayer.

In one aspect, the biopsy sample is derived from normal colon cells, or from adenomatous polyp. In another aspect, the biopsy sample is from a tumor.

In some other embodiments, a method of using the disclosed organoid to screen for bioactive compounds is disclosed. The method may include at least these steps: a) providing an organoid comprising a plurality of cells; b) staining the plurality of cells with a plurality of markers, wherein the plurality of markers binds to different components of the plurality of cells, c) applying a bioactive compound to some but not all organoids, and d) capturing images of the organoids (e) analyzing and comparing a plurality of features based on the images obtained in (d), said plurality of features being selected from the group consisting of cell type, morphology, proliferation, metabolism, cytoskeleton, cytotoxicity, and combination thereof; and f) determining the functionality of the bioactive compound based on the analysis and comparison performed in step (e) to determine whether the compound has certain bioactivity.

In one aspect, the organoid is loaded onto a multi-well plate in step (a). In another aspect, changes in the plurality of features are analyzed at a single cell level. Examples of bioactive compounds include but are not limited to proteins, nucleic acids, and small molecule chemicals. In some other embodiments, the bioactive compound is a small interfering RNA (siRNA).

In some other embodiments, a cell line derived from a human adenomatous polyp is disclosed, wherein the cell line is capable of forming the disclosed organoid comprising at least two compartments: a first compartment and a second compartment, wherein the first compartment comprises at least 10%, or 40%, or 50%, or 60%, or 80%, or 90%, or even 99% undifferentiated stem cells, and the second compartment comprises at least 60%, or 70%, or 80%, or 90% differentiated cells and less than 0.1%, or less than 0.5%, or less than 1% undifferentiated stem cells.

In another embodiment, the cell line is GiLA1 cell line (University of Arizona Tissue Bank number CORp86b).

### BRIEF DESCRIPTION OF THE FIGURES

The following figures form part of the present specification and are included to further illustrate aspects of the present invention.

FIG. 1 shows development and self-organization of colonic patient-derived colonic organoid monolayers. (A) Model depicting the workflow for the development of organoid monolayers. Patient biopsies are digested using collagenase to form a single cell suspension. Cells are then embedded in Matrigel and grown in 3D using a defined organoid growth medium. Organoids are expanded in 3D before seeding onto 2D imaging plates coated with a thin layer of Matrigel. Within 5 days, single cells self-organize to form a regularly patterned organoid monolayer. Organoid monolayers maintain homeostasis in this patterned state for up to 45 days. (B) Representative brightfield image of a single 3D colonic organoid. (C) Representative brightfield image of organoid monolayer. Left-automated image segmentation of nodes across a single well. Automated detection of nodes is shown in purple. Middle-sSingle node, zoom of left. Right-binary image of segmentation showing regular spacing of nodes across the culture. (D) Violin plot showing the distribution of spacing across organoid monolayers. Data represents quantification of 84 nodes (FIG. 1—source data 1). (E) Live cell images of self-organization over 5 days using organoids expressing H2B-iRFP670 for nuclear tracking. All scale bars represent 100 μM except C, left, which represents 1000 μM.

FIG. 2 shows characterization of Organoid Monolayer Compartments. (A) Representative FIG. 2D organoids stained for nuclei (blue), EdU (green), and ki67 (red). (B) Representative images of single-cell RNA fluorescence in situ hybridization (FISH) against Wnt target genes MYC and LGR5 in a single node. (C) Quantification of A showing the percent of positive cells in node or non-node regions, asterisks represent significance from paired t-test (FIG. 2—source data 1-2). (D) Quantification of images represented in B, mean fluorescence intensity (MFI) of target transcripts in each single cell is shown. Cells were separated based on presence in nodes vs non-nodes. Three to four biological replicates were performed. Data shown is from analysis of 64-224 cells binned from 16 technical replicates (FIG. 2—source data 3-5). Asterisks represent significance from Mann Whitney analysis, ****=p<0.0001 etc. All scale bars represent 100 μM.

FIG. 3 demonstrates that apoptosis Induces an ERK Signaling Wave that Instructs Cell Movement. (A) Model depicting how to interpret the ERK-KTR translocation reporter. Cells with mostly cytoplasmic ERK-KTR have high ERK activity (left, green arrow). Cells with mostly nuclear ERK-KTR signal have low ERK activity (right, red arrow). Scale bar represents 10 μM. (B) Representative images of the ERK-KTR active vs inactive. (C) Representative images showing a single ERK wave propagating from an apoptotic cell (white*). Heat map of the nuclear to cytoplasmic ratio of the ERK-KTR is shown in blue (low)/yellow (high). Scale bar in top image represents 100 μM. (D) Left: single-cell analysis of ERK activity over time after an apoptotic event. Heat maps are ordered from closest (top) to furthest (bottom) distance from the dying cell over time. ERK activity is shown (red, high ERK activity; blue, low ERK activity). Middle-single cell analysis of cell movement obtained from the same dataset shown on left. Change in distance towards the position of the apoptotic event is shown. Right-relative change in distance compared to the previous frame is shown. Data is represented from a single wave event. 455 cells were analyzed. (E) Representative single-cell traces comparing ERK activity (blue) and cell movement (red) at a given distance from the apoptotic cell is shown. (F) Left: duration of ERK signaling wave at a given distance from an apoptotic cell. Right-ERK-KTR activity at a given distance from an apoptotic cell (FIG. 3—source data 1). (G) Representative images of particle image velocimetry (PIV) of H2B-iRFP670 images after an apoptotic event. Organoids were treated with or without 5 μM Gefitinib. These data correspond with movie 3. Arrows indicate direction and amplitude of movement for the duration of the movie. Asterisk represent the position of the apoptotic cell. (H) Location of cleaved caspase three positive cells in monolayer over 24 hr. Data was acquired from 96 time points and is represented as the number of caspase positive cells in node or non-node area (FIG. 3—source data 2).

FIG. 4 shows that ERK Dynamics are Essential to Maintain Tissue Patterning in Organoid Monolayers. (A) Representative images of cell density variation in node vs non-node. Top left: H2B-iRFP670. Top right: ERK-KTR. Bottom left-binary nuclear segmentation showing high- and low-density regions. Bottom right: a spatial heat map of ERK activity over 16 hr in node and surrounding region. (B) Representative images of ERK activation after 100 nM phorbol 12-myristate 13-acetate (PMA) treatment for 20 min. Top-ERK-KTR before treatment with PMA. Bottom-ERK-KTR of the same node 20 min after PMA treatment. (C) Quantification of ERK-KTR activity after 30 min of 100 nM PMA treatment. Analysis from 255 single cells is shown (FIG. 4—source data 1). (D) Representative images of cell clustering following 100 nM PMA treatment. Top 4 panels show nuclear distribution across stitched image (top) and zoom (bottom) before and after PMA treatment. Bottom 4 panels show binary segmentation to clearly show loss of cell clustering following PMA treatment. (E) Histograms showing distance between nuclei with or without treatment with 100 nM PMA. Data is represented as nearest nuclei distance. Analysis of 3523 cells is shown (FIG. 4—source data 2). (F) Quantification of patterning loss before and after treatment with 100 nM PMA for 24 hr. Analysis of 53 images is shown (FIG. 4—source data 3). (G) Representative images of ERK-KTR activity after pulsing cells for 1 min with PMA followed by washout and chase for 24 hr. Top-all cells show high ERK activity with intact node. Bottom-node loss and resuppression of ERK activity after 24 hr. Asterisks represent significance from Mann Whitney analysis, ****=p<0.0001 etc. Scale bars represent 100 μM.

FIG. 5 shows expression of Oncogenic KRASG12V Induces ERK Activation, Increased Proliferation, Mitotic Abnormalities, and Tissue Patterning Loss. (A) Representative images of GiLA1 monolayers expressing H2B, Dox-inducible GFP-KRASG12V, and ERK-KTR before (top) and 7 hr after (bottom) 1 μg/ml of doxycycline. Scale bars represent 100 pm. (B) Quantification of KRASG12V induction after treatment with 1 μg/ml doxycycline for 16 hr. Analysis from 16 images over 54 timepoints is shown (FIG. 5—source data 1). (C) Quantification of ERK-KTR before and after 7 hr of doxycycline. Data is represented as the cytoplasmic to nuclear ratio of the KTR intensity (FIG. 5—source data 2). Analysis of 85-91 single cells is shown. (D) Quantification of the percent of mitotic cells with or without treatment with 1 μg/ml doxycycline for 16 hr (FIG. 5—source data 3). (E) Representative images of normal or abnormal mitotic events taken from experiments described in figures C and D. Scale bars represent 10 pm. (F) Quantification of abnormal mitotic events in cells treated with 1 μg/ml doxycycline for 16 hr. Data is represented as percent of total mitotic cells within the image. At least 58 images were analyzed for each condition. Images harbored 400-1200 cells (FIG. 5—source data 4). (G) Representative images of nodes in H2B and GFP-KRASG12V expressing cells with or without 1 μg/ml doxycycline for 7 days. Scale bars represent 100 pm. (H) Quantification of images shown in G (FIG. 5—source data 5). Data is represented as mean and SEM of at least 64 images harboring 400-1200 cells. Asterisks represent significance from Mann Whitney analysis, ****=p<0.0001 etc.

FIG. 6 shows Wnt and ERK Signaling Mutually Limit Each Other to Preserve Tissue Homeostasis. (A) Representative images of LGR5 mRNA in organoid monolayer after removal of Wnt3a for 72 hr. H2B-GFP is shown in white and LGR5 mRNA is shown in red. (B) Quantification of scRNA FISH for LGR5 in organoid monolayers after removal of Wnt3a from organoid media. Data are represented as the sum of LGR5 intensity per cell. Analysis of at least 386 cells is shown (FIG. 6—source data 1). (C) Representative images of ERK kinase activity after Wnt3a removal for 24 hr. Inverted nuclear intensity of ERK-KTR is shown. (D) Quantification of ERK kinase activity after removal of Wnt3a from organoid media from 0 to 48 hr following Wnt removal. Analysis of at least 287 cells is shown (FIG. 6—source data 2). (E) Left: Representative images of H2B (left) and ERK-KTR (right) with or without treatment with a dose response of Pyrvinium for 4 hr. Top right: quantification of ERK activity after treatment with the Wnt inhibitor pyrvinium for 4 hr. Analysis of at least 1844 cells is shown. Bottom right-quantification of node loss after treatment with a dose response of pyrvinium for 4 days. Analysis of 14-79 images is shown (FIG. 6—source data 3 and FIG. 6—source data 4). (F) Representative images of smRNA FISH for LGR5 in organoid monolayers after treatment with either 1 μM PD0325901 (MEKi) or 100 nM PMA for 24 hr followed by 24 hr of normal media. (G) Quantification of experiment described in F. Data is represented as histograms of the sum of LGR5 intensity per cell. Gates show percent of high and low LGR5+expressing cells. Analysis of 5014-11282 cells is shown. Asterisks represent significance from Mann Whitney analysis, ****=p<0.0001 etc. Scale bars represent 100 μM (FIG. 6—source data 5).

FIG. 7 shows apoptotic Cells Induce ERK Waves Which Limit the Stem Cell Compartment to a Defined Region. In the human colon, differentiated cells (light green) eventually undergo cell death (red cell) and are replaced by stem (dark green) and transit amplifying (lime green) cells. During homeostasis (top), apoptosis in the differentiated cell region triggers an ERK wave which propagates across the epithelium. This wave both triggers nearby cells to migrate (straight arrows) towards the site of apoptosis to maintain barrier function and also inhibits WNT signaling in order to maintain the correct proportion of stem, transit-amplifying, and differentiated cells. If ERK is inhibited (middle), the stem cell compartment expands and directional migration is lost. Conversely, if ERK is hyperactivated (bottom), cell movement becomes disorganized (wavy arrows) and spatially distinct cellular compartments are lost.

FIG. 8 illustrates organoid monolayer patterning in normal and tumor-derived tissues. (A) Representative images of LGR5 expressing murine small intestine organoid monolayer. Channels from left to right: LGR5-GFP (green) and phase contrast. (B) Images of organoid monolayers derived from normal (top) or tumor (bottom) derived colon tissue. Channels from left to right: DAPI (blue) and 13-catenin (red). (C) Representative image of another organoid monolayer derived from normal colonic tissue. Channels: DAPI (blue), Ki67 (green), and 13-catenin (red). (D) Representative images of Gila1 and five other tumor organoid monolayers prepared identically. No tumor lines, despite differences in major driver genes, showed tissue patterning (nodes) as seen with Gila1 polyp-derived or normal colonic organoid monolayers. Scale bars represent 100 μm. (E) Representative images of GiLA1 monolayers stained for either DCLK1 (tuft) or MUC2 (goblet) cell lineage markers. (F) Quantification of cell fates derived from data shown in E and FIG. 2.

FIG. 9 shows generation and validation of ErkKTR Organoids from Human Biopsies. (A) Model depicting the development of organoid monolayers expressing live cell reporters for kinase activity. (B) ERK-KTR activity in organoid monolayers. Cells were treated with 1 μM of Gefitinib (EGFRi), Erlotinib (EGFRi), PD0325901 (MEKi), or Trametinib (MEKi) for 24 hr. Average ERK activity across all cells over 24 hr is shown. Error bars represent standard deviation across three independent replicates.

FIG. 10 shows Cross-correlation Analysis of Cell Movement vs ERK Activity. Correlation is shown as a solid line and the median absolute deviation is shown as dashed lines.

FIG. 11 shows representative Images of Caspase 3 Dye Localization in node vs non-node areas. Red circle represents node area. Channels from left to right: H2B, ERK-KTR, cleaved caspase, and Brightfield. Scale bars represent 100 μm.

FIG. 12 shows the correlation Between Node Spacing and Wave Distance.

FIG. 13 shows loss of Patterning After KRASG12V Induction. GiLA1 monolayers harboring dox-inducible KRASG12V were allowed to pattern for 4 days and then treated with (Bottom) or without (Top) 1 μg/ml doxycycline every 24 hr for 14 days. Two representative images of H2B and KRASG12V are shown.

FIG. 14 shows pyrvinium Inhibits Wnt Signaling in Organoid Monolayers. Organoids were treated with either 3 μM pyrvinium to block Wnt signaling or 10 μM GSK-3 inhibitor to stimulate the Wnt pathway for 72 hr. Cells were fixed and stained for DAPI. Total GFP fluorescence across the monolayer was measured and normalized by mCherry control to obtain TopGFP measurements per condition. Significance between treatment groups was determined by t-test. Scale bar=100 μm.

FIG. 15 shows quantification of MYC Transcript Levels Following Treatment with either 100 nM PMA (RED) or 1 μM PD0325901 (MEKi-GREEN). Data is represented as sum intensity of the FISH probe in each cell. Gates were made against untreated control to display population shift.

### DETAILED DESCRIPTION

Disclosed here is a human 2D patient-derived organoid screening platform to study tissue patterning and kinase pathway dynamics in single cells. With the help of this system, it is discovered that waves of ERK signaling induced by apoptotic cells play a critical role in maintaining tissue patterning and homeostasis. If ERK is activated acutely across all cells instead of in wave-like patterns, then tissue patterning and stem cells are lost. Conversely, if ERK activity is inhibited, then stem cells become unrestricted and expand dramatically. This work demonstrates that the colonic epithelium requires coordinated ERK signaling dynamics to maintain patterning and tissue homeostasis. This work reveals how ERK can antagonize stem cells while supporting cell replacement and the function of the gut.

The human colon requires ongoing regeneration and renewal over a lifespan; this need relies on finely tuned communication between differentiated and stem cells. We show here that one way the colonic epithelium maintains both complexity and plasticity is through long-distance signaling driven by cell turnover. As cells differentiate and are eliminated from the epithelium, dying cells initiate an ERK wave that triggers local cell tropism towards the death event and stimulates differentiation of replacement cells. This process is an efficient way for the colon to maintain the correct proportions of stem and differentiated cells in distinct spatial compartments.

It is shown here that primary human colonic tissue can be expanded and transformed into monolayers that phenocopy key characteristics observed in the tissue of origin. Although the immune and stromal compartments are not included in this culture method, different compartments of stem and differentiated cells can be established and form unique physical properties depending on the source of the tissue. The role of the stromal and immune compartments in the maintenance of tissue patterning and their effect on ERK dynamics is an exciting area of future study. The epigenetic modifications have been highlighted as a major mechanism that dictates the memory of stem cells from their tissue of origin (Kim et al., 2010). It is possible that dynamic behavior in critical regulatory kinases like ERK can also play an important role in adult epithelial tissues in the continued maintenance of cell differentiation and tissue architecture of preprogrammed stem cells.

It is shown here that acutely inducing ERK signaling across all cells destroys the stem cell niche, suggesting that this mechanical feedback is essential to maintain stem cell homeostasis. Conversely, inhibition of the ERK pathway unleashes stem cells, resulting in aberrant expansion of the stem cell compartment. This result suggests that the dynamics and location of ERK signals help to maintain the balance between stemness and differentiation. Recent studies in animal models support a mutually inhibitory relationship between the Wnt and ERK pathways in the normal gut. In the murine small intestine, the ERK activity is low in the crypts of Lieberkühn and higher as cells move out of the crypt. Inhibition of para-crine Wnt signaling results in ERK hyperactivation in the crypt and loss of LGR5+ stem cells (Kabiri et al., 2018), suggesting that Wnt is suppressing ERK signaling as it maintains stemness. Inversely, if ERK1/2 is knocked out at embryonic stages in mice, stem cells within the crypt proliferate and expand dramatically, displaying a ˜two-fold increase in LGR5 and OLFM4 (Wei et al., 2020). This phenotype is strikingly similar to what was observed after chemical inhibition of the ERK pathway in our organoid monolayers. In murine systems, oncogenic activation of ERK driver genes leads to decreases in the stem cell population (Riemer et al., 2015; Leach et al., 2021; Reischmann et al., 2020), which is similar to what we observed using an inducible KRASG12V allele. During hair follicle development, a well-established patterning model system, EGFR is essential and serves to limit cell proliferation and stem cell numbers through attenuation of Wnt signaling (Tripurani et al., 2018). β-catenin is also essential for follicle formation, and hyperactivation of the Wnt pathway results in de novo follicle expansion (Närhi et al., 2008). Together, these studies support the hypothesis that these two pathways balance one another to maintain tissue homeostasis.

ERK was activated via four separate methods in GiLA1 monolayers to assess its role in patterning maintenance: (1) PMA treatment (2) Expression of oncogenic KRAS (3) Removal of Wnt 3 a (4) Inhibition of Wnt via pyrvinium. Although activation of ERK occurred within hours in all four conditions, only PMA triggered rapid node loss, whereas other methods took days to induce patterning defects. We suspect this is due to the role of PKC (activated by PMA) in cell migration. PMA treatment most likely induced ERK activity and migration, leading to a previously described positive feedback loop (Hino et al., 2020). The presently disclosed organoid monolayers lack stromal and immune cells, suggesting that the maintenance of patterning is most likely driven by signaling between epithelial cells rather than stromal or immune cell interactions with the colonic epithelium.

In another embodiment, the Wnt and ERK pathways are commonly hyperactivated during the development of colon cancer. This work unveils a relationship between these two pathways that maintains homeostasis in precancerous tissue. The aggressiveness of colon cancer is ranked in part by the loss of patterning that has occurred within the tissue (Chang et al., 2014; Vogelstein et al., 1988). Our work highlights the ERK and Wnt pathways as maintainers of patterning and therefore restrictors of dysplasia. One hypothesis is that Wnt pathway activation results in the suppression of ERK during early colon tumor development. Hyperactivating mutations such as oncogenic KRAS could be a selective response to Wnt activation. The result would be both the Wnt and KRAS pathways, which normally oppose one another, becoming hyperactivated together. This mutational combination, also requiring p53 suppression (Serrano et al., 1997), is seen in ˜30% of colon tumors and results in highly mobile, proliferative, and adaptive clones.

The mutation frequency in the normal epithelium is far too high for DNA repair alone to explain how humans prevent the expansion of cancerous cells over a lifetime (Rozhok and DeGregori, 2015). Question remains as to how normal tissue prevents the takeover of hyperproliferative clones throughout life. Pathways high-lighted as tumor-driving can also play a vital role in tumor prevention within normal tissue. In human cell lines, oncogenic mutations that trigger sustained ERK signaling result in extrusion carried out by normal neighbors in cell mixing experiments. This surveillance, mediated by normal cells, is driven by the ERK waves (Aikin et al., 2020) and is an example of normal cells using a pathway described regularly as oncogenic to prevent the establishment of transformed clones. Similarly, epithelial cells rely on Wnt signaling to extrude precancerous cells that have acquired Wnt pathway activating mutations. (Brown et al., 2017). The present work highlights how Wnt and ERK can regulate the size and spacing of cellular compartments in pre-cancerous tissue and shows how delicately these pathways are balanced.

The articles “a,” “an” and “the” are used to refer to one or more than one (i.e., to at least one) of the grammatical object of the article.

The terms “comprise”, “comprising”, “including” “containing”, “characterized by”, and grammatical equivalents thereof are used in the inclusive, open sense, meaning that additional elements are not expressly mentioned but may be included. It is not intended to be construed as “consists of only.”

The terms “polypeptide,” “peptide” and “protein” may be used interchangeably in this disclosure. The terms “oligonucleotide,” and “polynucleotide” may also be used interchangeably in this disclosure.

The term “organoid” refers to a miniaturized version of an organ produced in vitro in two or three dimensions by a group of cells that shows realistic micro-anatomy similar to an organ.

The instant disclosure is further described by the following items:

Item 1. An organoid comprising a plurality of cells derived from a human, said organoid comprising at least two compartments: a first compartment and a second compartment, wherein the first compartment comprises at least 10% undifferentiated stem cells, and the second compartment comprises at least 60% differentiated cells and less than 0.1% undifferentiated stem cells.

Item 2. The organoid of Item 1 wherein the plurality of cells is derived from normal colon cells, or from adenomatous polyp.

Item 3. The organoid of any of preceding items, wherein the plurality of cells is of tumor origin and therefore lacks contact inhibition.

Item 4. The organoid of any of preceding items, wherein the plurality of cells forms a monolayer on a tissue culture plate.

Item 5. The organoid of any of preceding items, wherein Erk kinase activity is lower in the first compartment than Erk kinase activity in the second compartment.

Item 6. A method for preparing an organoid, comprising
a) digesting a biopsy sample obtained from human intestine with collagenase to form a single cell suspension,
b) transferring single cells in the suspension from step (a) onto one or more wells of a multi-well plate, said one or more wells being coated with extracellular matrix, and
c) allowing single cells to grow and self-organize to form a 2D organoid monolayer.

Item 7. The method of Item 6, wherein the biopsy sample is derived from normal colon cells, or from an adenomatous polyp.

Item 8. The method of any of Items 6-7, wherein the biopsy sample is of tumor origin.

Item 9. A method of screening for bioactive compounds, using the organoid of Item 1, comprising
a) providing an organoid comprising a plurality of cells;
b) staining the plurality of cells with a plurality of markers, wherein the plurality of markers binds to different components of the plurality of cells;
c) applying a bioactive compound to some but not all organoids;
d) capturing images of the organoids;
e) analyzing and comparing a plurality of features based on the images obtained in (d), said plurality of features being selected from the group consisting of cell type, morphology, proliferation, metabolism, cytoskeleton, cytotoxicity, and combination thereof; and
f) determining the functionality of the bioactive compound based on the analysis and comparison performed in step (e).

Item 10. The method of Item 9, wherein the organoid is loaded onto a multi-well plate in step (a).

Item 11. The method of any of Items 9-10, wherein changes in the plurality of features are analyzed at a single cell level.

Item 12. The method of any of Items 9-11, wherein the bioactive compound is selected from the group consisting of proteins, nucleic acids, and small molecule chemicals.

Item 13. The method of any of Items 9-12, wherein the bioactive compound is small interfering RNA (siRNA).

Item 14. A cell line derived from a human adenomatous polyp, wherein said cell line is capable of forming the organoid of Item 1.

Item 15. The cell line of Item 14, wherein said cell line is GiLA1 cell line (University of Arizona Tissue Bank number CORp86b).

Item 16. A cell line derived from a human adenomatous polyp designated GiLA1 cell line (University of Arizona Tissue Bank number CORp86b).

All references cited in this disclosure, including but not limited to patents, patent applications and published papers, are hereby incorporated by reference into this disclosure.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1 Methods

The normal or tumor tissues from endoscopic ultrasound-guided fine-needle aspiration biopsies (EUS FNAs), or core needle biopsies were collected from consented patients by the (Tissue Acquisition and Repository for Gastrointestinal and HEpaTic Systems [TARGHETS], IRB 1909985869) facility located in the Arizona Health Science Center. Primary colonic organoid cell lines were anonymized by the Tissue Acquisition and Cellular/Molecular Analysis Shared Resource (TACMASR) at the University of Arizona Cancer Center. TACMASR is an on-campus biorepository to procure, store and retrieve bio-specimens in a form that is deidentified and protects the privacy of the donors and confidentiality of the data collected. The individuals from whom the cells originated were resection patients at Banner University Medical Center. All research participants in this proposal receive the cells with de-identified and anonymous labels that cannot trace back to the individual or their families from which they came. Thus, no one involved in this study can access the subject's identities. Therefore, the study is exempt from being considered human subject research. Biopsy tissues were transported in 50 ml conical tubes containing collection media (Advanced DMEM/F-12 [Invitrogen 12634028] supplemented with 2 mM GlutaMax, 10 mM HEPES, 0.25 mg/ml Amphotericin B, 10 mg/ml Gentamycin, 1% Kanamycin, N-2 media supplement [Invitrogen 17502048], B-27 Supplement Minus Vitamin A [Invitrogen 12587010], 1 mM N-Acetyl-L-cysteine [Sigma A9165], 10 nM Nicotinamide [Sigma Aldrich; #N0636], 2.5 μM CHIR99021 [Tocris-Fisher, 4423; Apexbio Technology, A3011], and 2.5 μM Thiazovivin. Tissues were then washed, minced, and cryopreserved in organoid freezing media 70% seeding media [see next section], supplemented with 20% FBS, 10% DMSO, and 2.5 μM Thiazovivin) at the BioDROids core facility, located in the University of Arizona Cancer Center. Frozen tissues were prepared into organ-oids by thawing tissue pieces, mincing, and incubation with 1 mg/ml collagenase type 3 in phosphate-buffered saline (PBS) on a shaker for 10-25 min at room temperature. Cells were then removed from collagenase, washed with PBS, embedded into 100% Matrigel, and cultured in seeding media for 7 days. After organoids became established, low passage aliquots were cryopreserved in freezing media or infected with lentiviral constructs.

Colorectal Cancer Organoids from the Patient-Derived Models Repository (PDMR)

A set of 16 colorectal cancer organoid models were purchased from the National Cancer Institute Patient-Derived Models Repository (PDMR; NCI-Frederick, Frederick National Laboratory for Cancer Research, Frederick, MD; https://pdmr.cancer.gov/). The set were selected on the basis of microsatellite stable and APC mutation-negative status (seven organoids) and a set of microsatellite stable and APC mutation-positive organoids were matched to the former set on the basis of age, gender, location, and stage. Two organoids from microsatellite unstable tumors were also selected, one APC mutation-negative and the other APC mutation-positive. In the present study, APC mutation-negative organoids 555926-031 R-V1-organoid and 624824-186 R-V1-organoid, APC mutation-positive organ-oids 276233-004 R-V1-organoid, 451658-271 R-V1-organoid and 722911-139 R-V1-organoid, and the microsatellite unstable organoid 616215-338 R-V2-organoid were expanded in Matrigel and organoid media according to the PDMR's published protocols.

### Growth Conditions of 3D and Organoid Monolayers Complete LWRN Media

Advanced DMEM/F-12 (Invitrogen 12634028) supplemented with 2 mM GlutaMax, 10 mM HEPES, N-2 media supplement (Invitrogen 17502048), B-27 Supplement Minus Vitamin A (Invitrogen 12587010), 1 mM N-Acetyl-L-cysteine (Sigma A9165), 2500 units/mL Penicillin and 2.5 mg/mL streptomycin (0.05 mg/mL), 50% L-WRN Conditioned Media (Sugimoto et al., 2018), 100 ng/ml huEGF (R&D 236-EG-01M), 500 nM A 83-01 (Tocris-Fisher, 29-391-0; APexBio-Fisher, 501150476), 10 μM SB 202190 (Tocris-Fisher, 12-641-0), 100 μg/ml Primocin, and 10 mM Nicotinamide (Sigma Aldrich; #N0636).

### Lentiviral Infection of 3D Organoids

3×106 HEK 293T cells were seeded in a 10 cm plate and transfected the following day using 500 μL of opti-MEM, 30 μL Gene Juice (Sigma #70967), 5 μg lentiviral construct, 3.25 μg psPAX2 (addgene #12260), and 1.75 μg pMD2.G (addgene #12259). After 24 hr, fluorescence was assessed, and media was collected for 3 days followed by centrifugation and filtration using 0.45 μM syringe filter. Lentiviral media was then kept at 4° C. before 100×concentration using LentiX concentrator (Takara #631232) and resuspension of virus in organoid seeding media; viral media was then stored at −80° C. for up to 3 months. For infection of organoids, 1×105 primary epithelial cells were harvested from 3D domes, washed, and trypsinzed as described previously. Cells were then counted using a hemocytometer and 40 K organoid cells were plated in suspension onto a 48 well plate and diluted 1:1 in vial media containing 8 mg/ml polybrene. Next, organoids were placed at 37° C. for 1 hr the before centrifugation at 600 g for 1 hr at 32° C. Organoids were then harvested, washed, and embedded into 90% Matrigel and cultured in seeding media for 24 hr. Cells were then grown in complete LWRN for 3 days before fluorescence was assessed and >50% infection was achieved. Media was aspirated and organoids were resuspended in 500 ul trypsin. After dissociation, cells were mixed 5-10 times using a p200 pipette tip and passed through a 100 μm filter to ensure isolation of single cells.

Preparation of Organoid Monolayers from 3D Cultures

SCREENSTAR 384-well black plates (Grenier #781866) were coated with ice cold Matrigel (CB40230C) diluted 1:40 in Serum-free Advanced-DMEM F12 media (SFM) for 1 hr. Monolayers were prepared using our previously described protocol (Thorne et al., 2018; Sanman et al., 2020). Briefly, cells were removed from 3D Matrigel and resuspended in ice cold SFM and washed three times using ice cold SFM. Organoids were then dissociated by resuspension in Trypsin+10 μM Y-27 for 4 min followed by quenching in DMEM supplemented with 10% FBS and washing. Coating media was removed from 384-well plates and organoids plated at 7000 cells/well after counting using hemocytometer and cultured for 24 hr. Seeding media was then removed and replaced with complete LWRN media and changed daily for 7 days until cultures became confluent and tissue patterning was observed.

### High Content Imaging of Organoid Monolayers

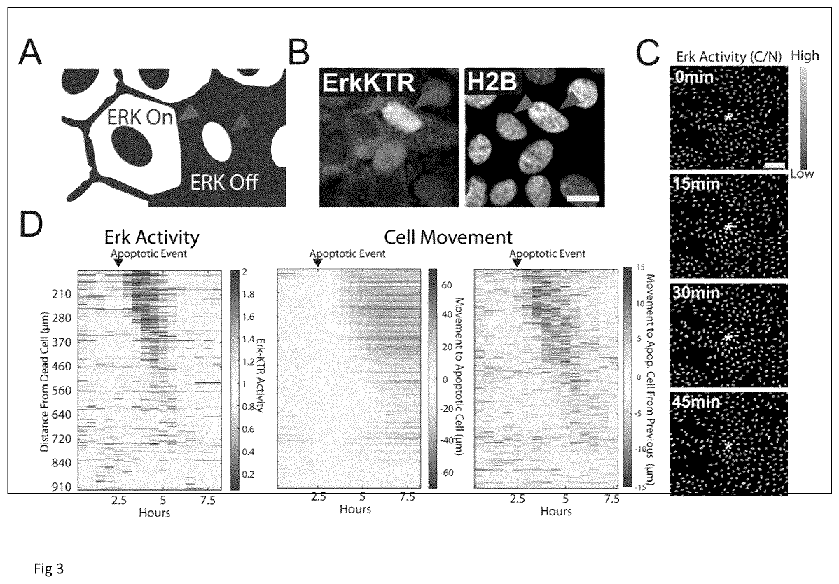

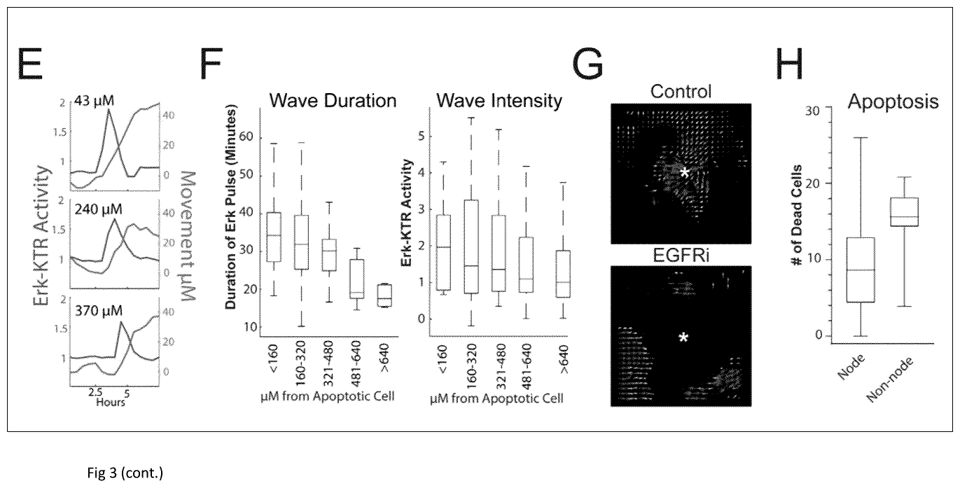

384-well plates were imaged with fluorescent microscopy on a Nikon Eclipse TI2 automated microscope. For quantification of wave dynamics, organoids were imaged every 10-30 min. Tracking and segmentation of single cells in time lapse images in FIG. 2 was performed using the MATLAB program p53Cinema (Reyes et al., 2019). Analysis of the extracted data was performed in MATLAB. For the ERK activity heat maps in FIG. 3, images of the ERK-KTRmRuby2 and the H2B-iRFP670 were captured every 7 min for 24 hr. For each frame, individual nuclei were segmented using a MATLAB script developed in-house that identifies nuclei based on the H2B-iRFP670 images. The cytoplasmic ERK-KTRmRuby2 signal was obtained by creating a two pixel wide annulus surrounding the nucleus and averaging the intensity. Cells with active ERK were identified as having a mean ERK-KTR cytoplasmic signal>the mean ERK-KTR nuclear signal. The density-based spatial clustering of applications with noise algorithm (dbscan, MATLAB, 2021b) was performed on the centroids of ERK active cells to identify spatial clusters of cells with active ERK. A boundary was drawn around the spatial clusters using the boundary algorithm in MATLAB. The ERK positive regions of each frame were summed up to generate the heat map in MATLAB.

### Example 2 Generation of the GiLA1 Organoid Line

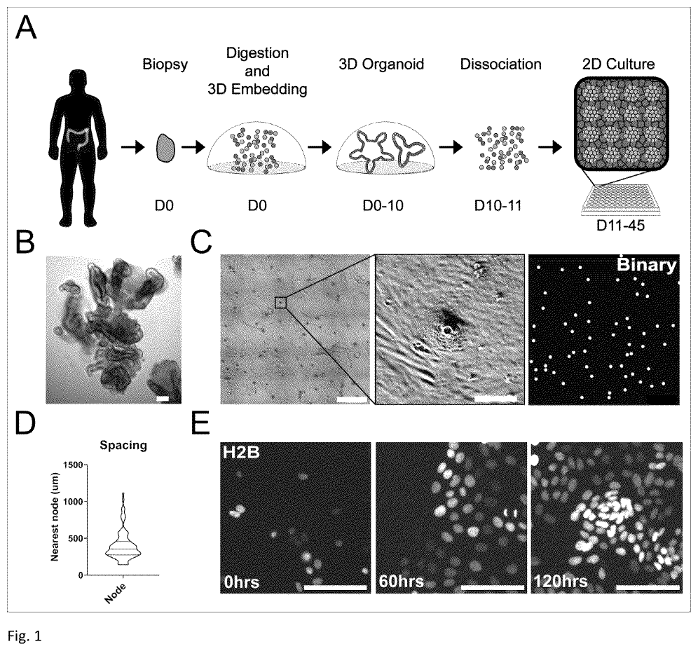

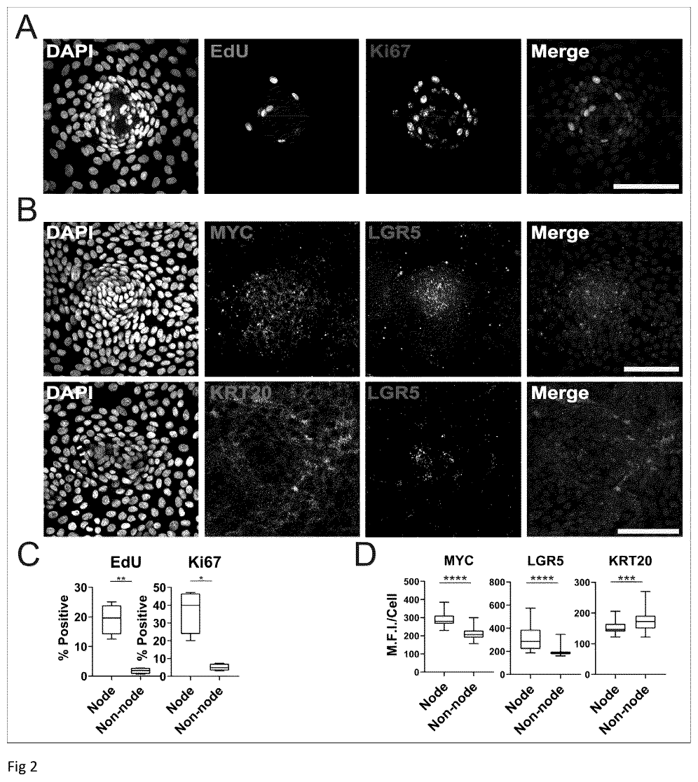

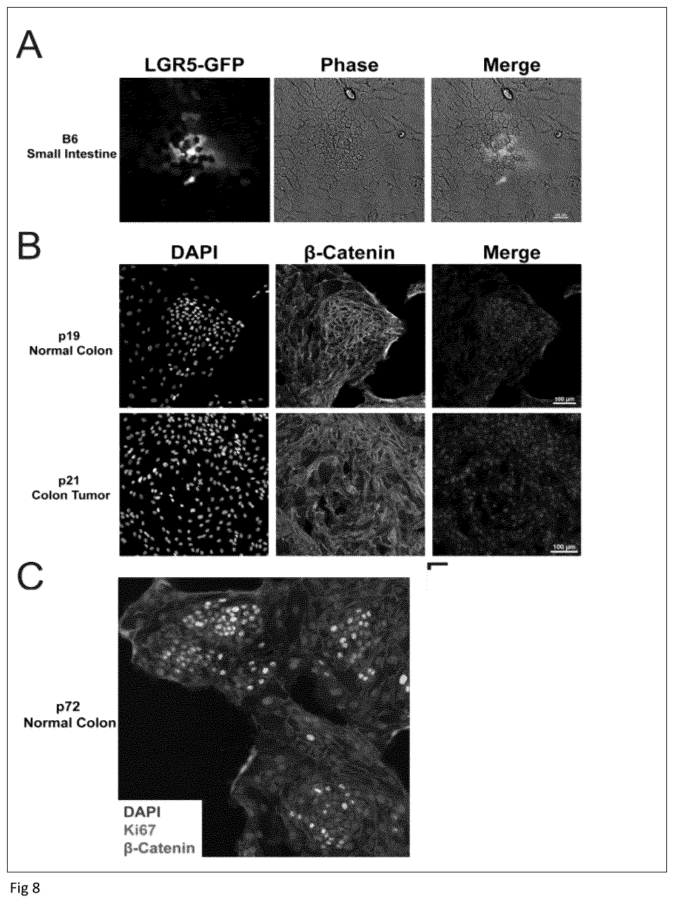

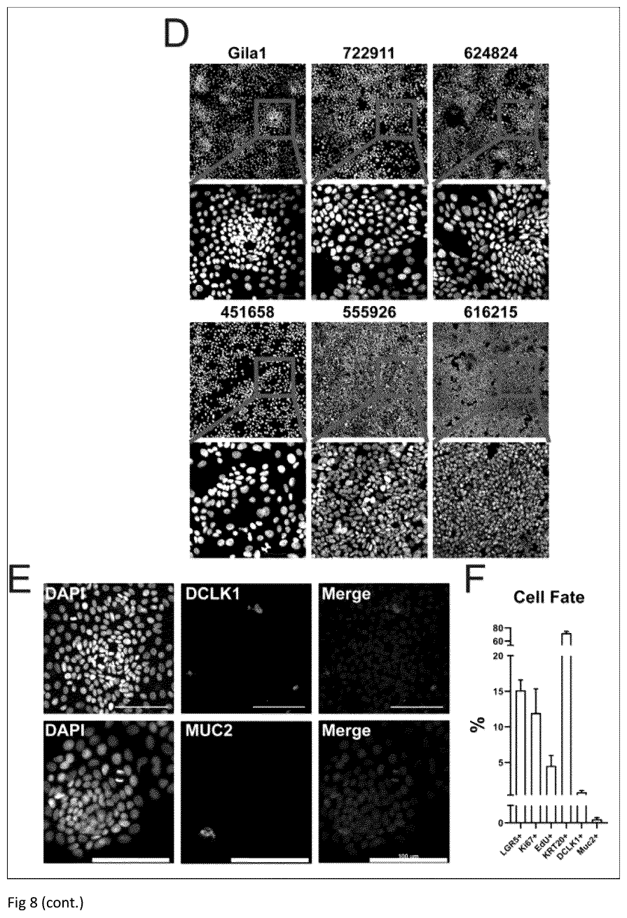

To study single-cell dynamics in the human colon, in conjunction with the University of Arizona Cancer Center organoid resource (BioDROid), colonic normal, adenomatous polyp, and carcinoma samples were collected, and cultured as 3D patient-derived organoids. They were then expanded in 3D and transferred onto a thin layer of extracellular matrix after dissociation into single cells. After 4 days, confluent monolayers harbored phenotypically distinct clusters and were considered fully established (FIG. 1A and C). Strikingly, even starting as a random dispersion of single cells, normal colonic organoids developed localized compartments of densely packed cells (nodes) that were surrounded by less dense cells. These nodes resembled those we observed using the murine model system (Thorne et al., 2018), with LGR5+ stem cells located specifically within densely compacted cell compartments (FIG. 8A). Murine small intestinal organoid monolayers and normal human colon monolayers rarely reached confluence and instead reached a homeostatic state and maintain nodes at subconfluency (FIG. 8A-C).

A polyp-derived 2D organoid line (referred to here as GiLA1 for Gastrointestinal Line, Arizona 1) formed regularly spaced nodes, quickly reached confluence within 72 hr, and could maintain node structures for 45 days or more. When GiLA1 single cells were seeded onto an ultra-thin layer of Matrigel, organoids formed monolayers by 3 days and began to spontaneously form regularly spaced compact compartments reminiscent of colonic crypts within 3-4 days (FIGS. 1C and D). A tumor organoid derived from a micro satellite stable (MSS) invasive colon adenocarcinoma (p21T) was grown in 2D and displayed different morphologies compared to normal, polyp, and the murine small intestine (FIG. 8B). p21T proliferated but failed to form nodes and instead formed uniform, disorganized monolayers (FIG. 8B). To date, across multiple patient-derived organoid lines, formation of nodes in normal and polyp-derived organoids have been observed; however, we fail to observe nodes in multiple adenocarcinoma organoids. Across five colorectal patient lines obtained from the PDMR, no patterning was observed, and all lines formed confluent monolayers. (FIG. 8D). Because of its unique ability to form both complete monolayers and distinct cellular compartments, the GiLA1 organoid line was used for the remainder of the described experiments.

### Example 3 Self-Organization of Organoid Monolayers

A striking aspect of the organoid monolayers is that they appear to self-organize into node/non-node compartments. To capture the establishment of these structures over time, time-lapse microscopy was used on GiLA1 organoid monolayers expressing H2B-iRFP670 to observe the formation of distinct cellular compartments over 5 days. Cells initially exhibited high levels of motility after attachment to the extracellular matrix (ECM) to form 5-10 cell clusters. After this, cells became hyperproliferative from 48 to 72 hr after seeding to form a complete monolayer. Finally, morphologically distinct cellular compartments were formed by 96 hr (FIG. 1E). These 2D monolayers can be readily grown in 384-well imaging plates making this model particularly amenable to high-content imaging and quantitative-image analysis. Together, these data show that patient biopsies from the human colon can fully self-organize into patterned monolayers that maintain long-term tissue homeostasis.

### Example 4 Spatially Distinct Stem and Differentiated Cell Compartments

The GiLA1 organoid line was further characterized by determining the cell types in organoid mono-layers and their location with respect to the nodes. Two proliferative markers, Ki-67 and EdU, revealed that proliferative cells were much more abundant within the compartments of densely packed cells of the organoid monolayers (FIGS. 2A and C), suggesting the nodes are a collection of transit amplifying-like cells and possibly stem cells. To determine if nodes harbored stem cells, single-molecule RNA fluorescence in situ hybridization (smRNA FISH) was performed against Wnt target genes and stem cell markers, LGR5 and MYC. LGR5 and MYC expressed at higher levels in nodes compared to non-nodes (FIGS. 2B and D). Cells expressing the differentiated-colonocyte marker KRT20 were primarily located in non-node regions and were mutually exclusive from LGR5 high compartments (FIGS. 2B and D). Organoids harbored a majority of colonocytes, whereas tuft, goblet, stem, and proliferative cell markers were observed at low levels indicating normal cell fates were maintained in GiLA1 monolayers (FIGS. 8E and 8F). These data show that patterned organoid monolayers contain cells positive for stem, proliferative, and differentiated cell markers.

### Example 5 Apoptosis-Induced ERK Waves Instruct Cell Movement

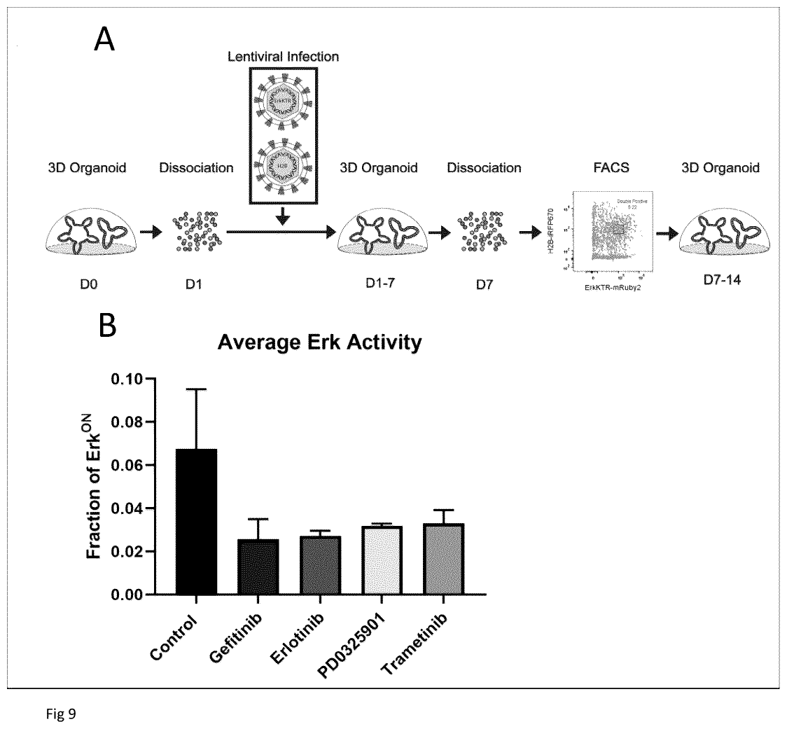

MAPK/ERK signaling is a critical regulatory pathway involved in maintaining homeostasis of epithelia in the gut (Wei et al., 2020). To assess ERK activity in human organoid monolayers over time, we utilized a well-characterized ERK kinase translocation reporter (ERK-KTR) (Regot et al., 2014). The ERK-KTR utilizes a bipartite nuclear localization signal (NLS) and nuclear export signal (NES) to transform ERK kinase activity into a nuclear to cytoplasmic shuttling event that is easily quantified by microscopy in organoid monolayers (FIG. 3A). We transduced the GiLA1 organoid line with a H2B-iRFP670 nuclear marker and ERK-KTR-mRuby2 reporter to perform nuclear and cytoplasmic segmentation and calculate nuclear-to-cytoplasmic ratios of the ERK-KTR (FIG. 3A, FIG. 3B and FIG. 9A). In fully developed organoid monolayers, a-twofold reduction of the ERK-KTR was observed after treatment with inhibitors of EGFR or MEK across all cell types, suggesting the KTR is actively representing ERK signaling and is EGFR dependent (FIG. 9B). Interestingly, when GiLA1 monolayers were imaged using time lapse microscopy, ERK activation was observed originating at focal points within the confluent monolayer that propagate outward in a wave-like pattern. These waves did not overlap and were observed routinely during homeostasis (FIG. 3C, FIG. 3). Imaging of monolayers using brightfield and caspase 3 dye revealed apoptotic cells that were being extruded from the monolayer to be the focal source of wave activation (FIG. 3).

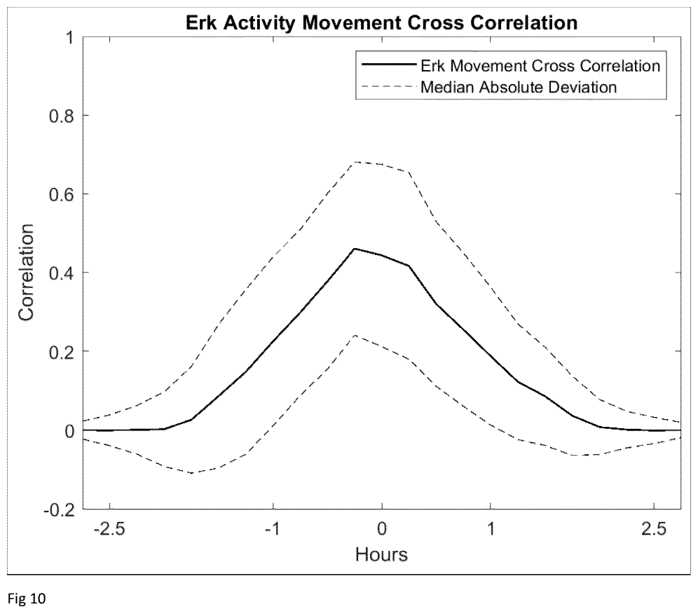

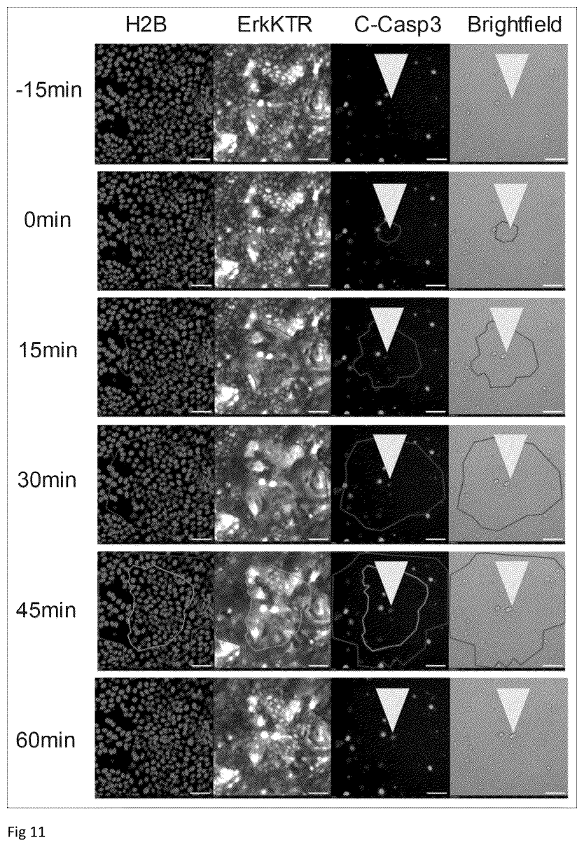

To determine the properties of the ERK waves, about 450 cells were tracked and their ERK activity was assessed over time following an apoptotic event (FIG. 3D, left panel and FIG. 3E). During the apoptotic event, the cells immediately adjacent to the apoptotic cell moved away from the apoptotic cell slightly. The apoptotic event was followed by a wave of ERK activity that traveled approximately 450 μM over a period of 95 min for an average speed of −4.7 μM/min. In addition to the ERK signaling wave, we noticed motility of the cells surrounding the apoptotic cell towards the site of apoptosis. Tracking single cells revealed a striking correlation between intensity/duration of ERK activity and movement of cells surrounding the apoptotic cell in the direction of the apoptotic cell (FIG. 3D, two right panels and 3E). As the ERK wave propagated outward from apoptotic cells, it dissipated in both duration and intensity (FIG. 3F). Particle image velocimetry (PIV), a method used to quantify the flow of image pixels over time (Adrian, 1991), confirmed collective cell migration towards the apoptotic cell (FIG. 3G), suggesting that ERK waves instruct migration of surrounding cells as shown in previous studies (Gagliardi et al., 2020). Cells treated with the EGFR inhibitor Gefitinib did not induce ERK waves or display tropism towards dying cells, suggesting that EGFR-mediated ERK signaling is required for cell movement in this context (FIG. 3G). Tracking of single cells (FIG. 3E) and cross correlation analysis (FIG. 10) revealed that ERK activity preceded cell movement, suggesting that ERK waves instruct cell movement. To determine whether distinct apoptotic and proliferative compartments exist in human organoid monolayers and investigate if cell death was restricted to a compartment distinct from proliferative nodes, we imaged mature living organoid monolayers in the presence of cleaved caspase 3 dye, a marker for apoptosis. It is observed that the emergence of an apoptotic cell was more likely in differentiated cells in non-node compartments (FIG. 3H and FIG. 11). Taken together, these data show that ERK waves induced by apoptotic cells instruct surrounding cells to migrate toward the apoptotic site.

### Example 6 ERK Waves Instruct Patterning in Organoid Monolayers

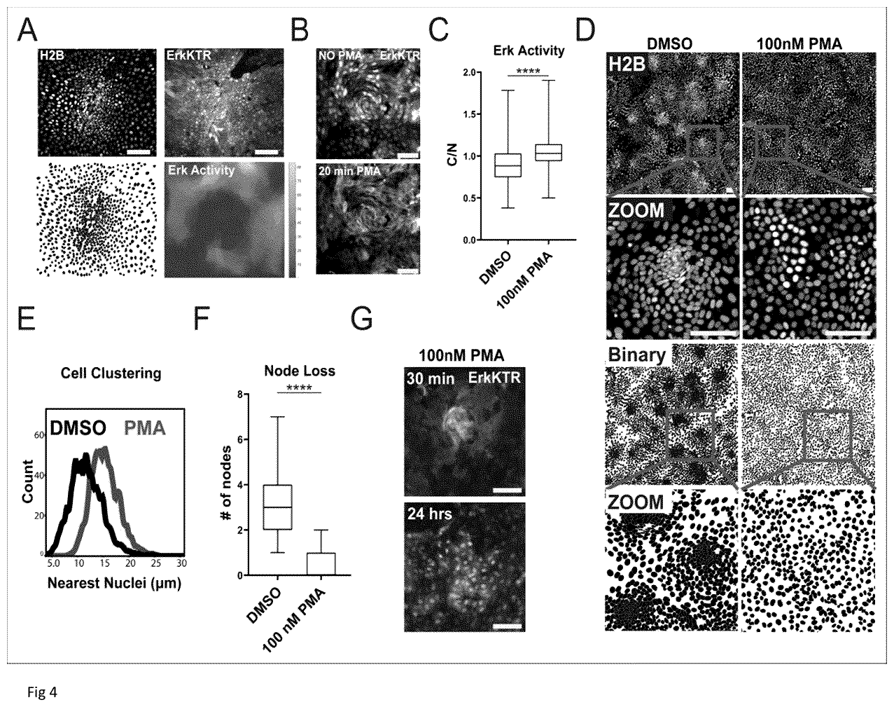

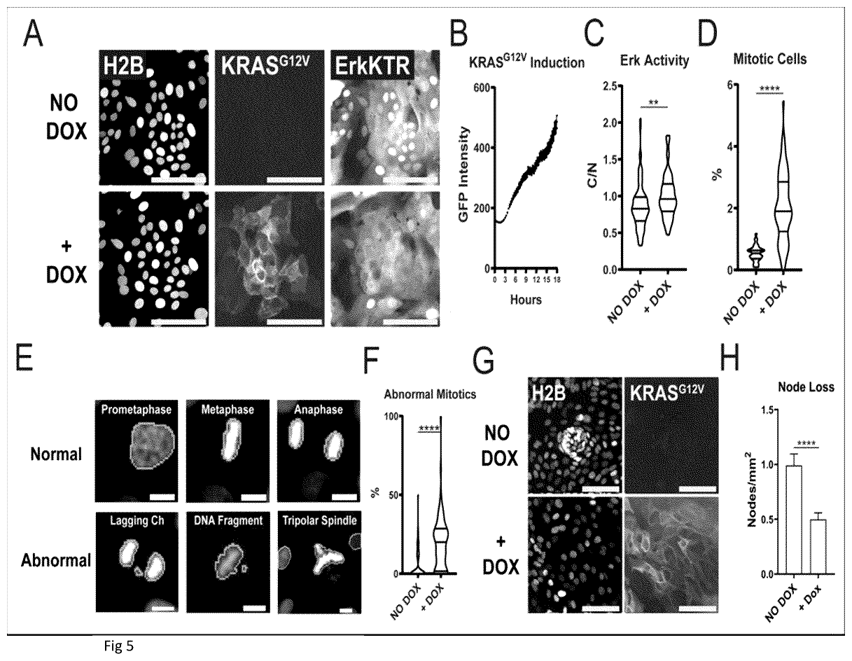

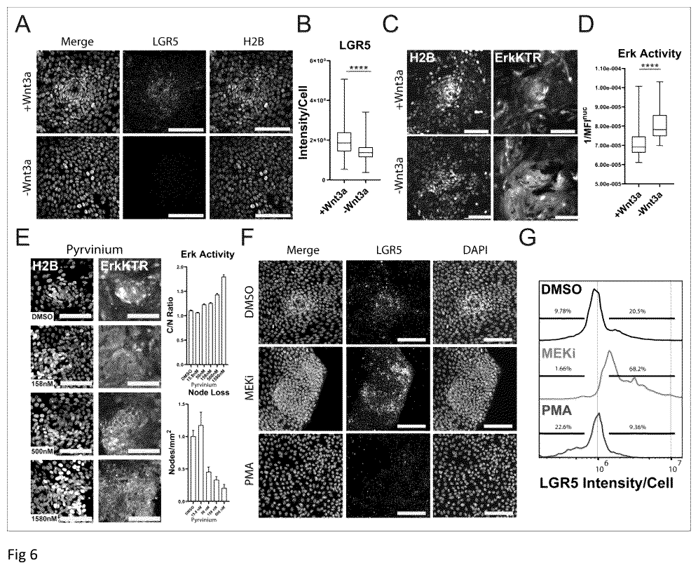

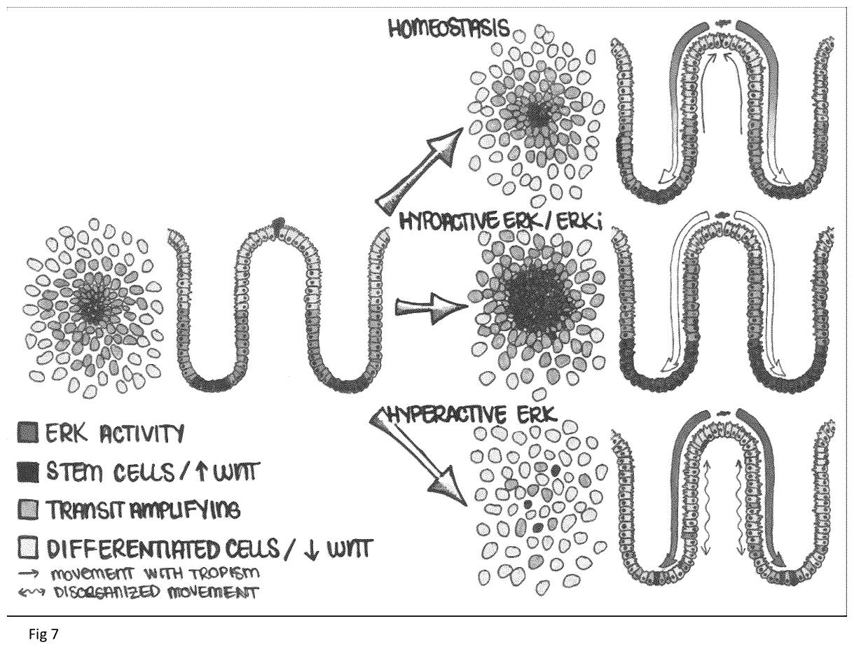

To determine whether ERK activity was increased in the differentiated compartment in GiLA1 monolayers, we created a time lapse movie of a dense node compartment surrounded by less dense, non-node cells over a 16 hr period. Nuclear to cytoplasmic ratio of the ERK-KTR was measured at the single cell level over time. A threshold for active ERK cells was then combined with spatial clustering to identify ERK-active regions. High ERK regions were then superimposed from each imaging timepoint to create a single heat map for regional ERK activity over time. It was observed that cells in non-node compartments were more mobile and displayed more active ERK signaling (FIG. 4A). Together, these data show that apoptotic events that cause ERK waves are mostly localized in spatially distinct differentiated cell compartments.

Since ERK waves are largely restricted to the post-mitotic, differentiated compartments in our gut organoid monolayer model, we hypothesized that ERK waves may help regulate the patterning observed in the GiLA1 monolayers by restricting the stem cell compartment. To test this hypothesis, we asked if global activation of ERK activity could disrupt the patterning of the organoid monolayers. a well-established activator of the ERK pathway, phorbol 12-myristate 13-acetate (PMA) was used to acutely activate ERK signaling across the monolayer. Following treatment with 100 nM PMA, immediate hyperactivated ERK was observed in all cells (FIGS. 4B and C). Monolayers showed a remarkable loss of patterning after PMA treatment as evidenced by almost complete loss of cell clusters by 24 hr post-treatment (FIG. 4D-F). To determine if nodes re-formed once ERK activity returned to baseline, we treated GiLA1 monolayers with a pulse of 100 nM PMA for 1 min. The pulse PMA treatment caused ERK activation in all cells after 30 min, followed by node loss. Notably, ERK levels returned to normal after PMA treatment, but nodes did not re-form by 48 hr (FIG. 4G). These data suggest a potential role for ERK waves in regulating the ratio of stem to differentiated cells in organoid monolayers.

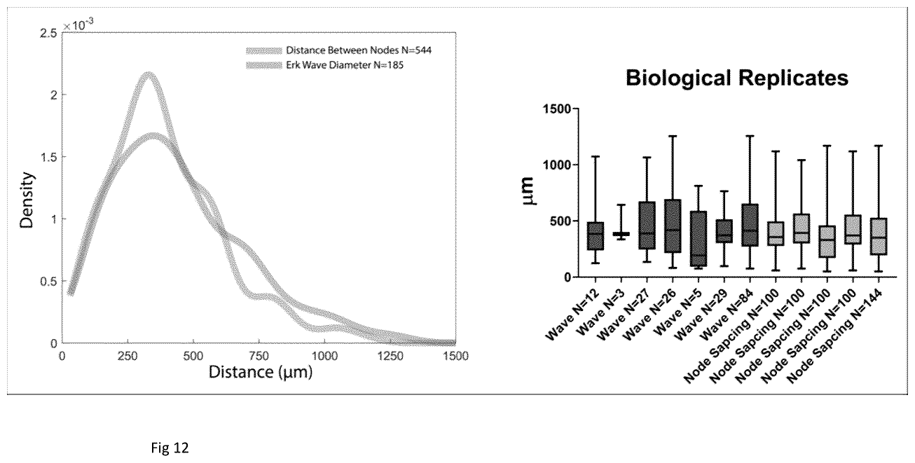

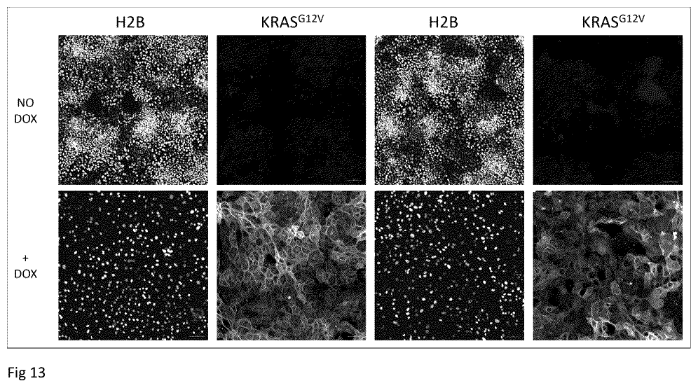

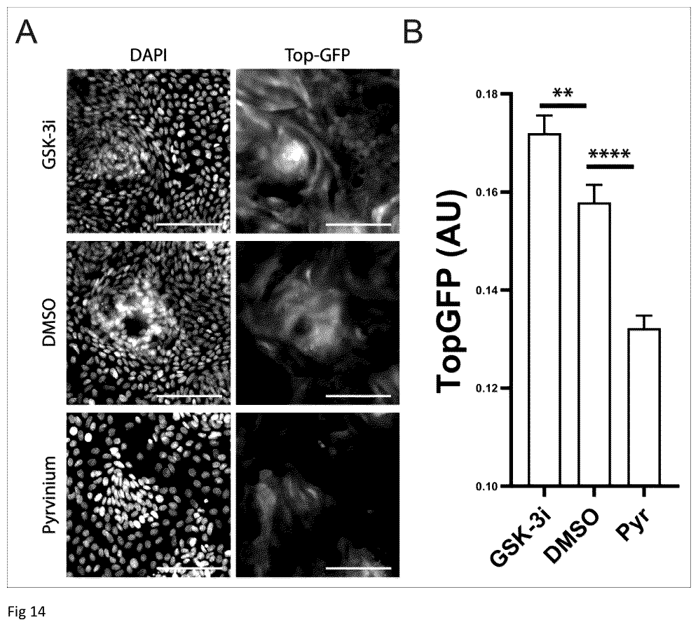

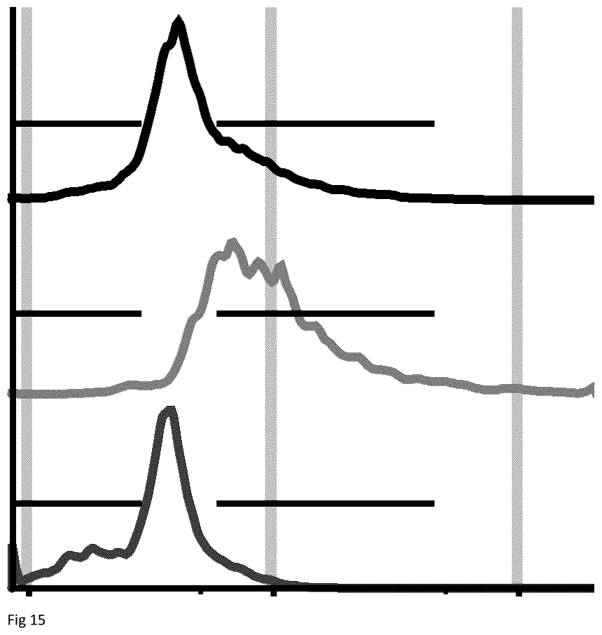

In support of this hypothesis, a correlation was observed between the average distance (−399 μm) between neighboring proliferative compartments and the average distance (−447 μm) covered by the apoptosis-induced ERK wave (FIG. 12A). This result was maintained across multiple biological replicates with high fidelity (FIG. 12B).

### REFERENCES

All references listed below are hereby incorporated by reference into this disclosure.
Adrian R J. 1991. Particle-imaging techniques for experimental fluid mechanics. Annual Review of Fluid Mechanics 23:261-304.
Aikin T J, Peterson A F, Pokrass M J, Clark H R, Regot S. 2020. MAPK activity dynamics regulate non-cell autonomous effects of oncogene expression. eLife 9:e60541.
Ashton G H, Morton J P, Myant K, Phesse T J, Ridgway R A, Marsh V, Wilkins J A, Athineos D, Muncan V, Kemp R, Neufeld K, Clevers H, Brunton V, Winton D J, Wang X, Sears R C, Clarke A R, Frame M C, Sansom O J. 2010. Focal adhesion kinase is required for intestinal regeneration and tumorigenesis downstream of wnt/c-myc signaling. Developmental Cell 19:259-269.
Brown S, Pineda C M, Xin T, Boucher J, Suozzi K C, Park S, Matte-Martone C, Gonzalez D G, Rytlewski J, Beronja S, Greco V. 2017. Correction of aberrant growth preserves tissue homeostasis. Nature 548:334-337.
Carver J, Dexheimer T S, Hsu D, Weng M-T, Smith J L, Guha R, Jadhav A, Simeonov A, Luo J. 2014. A high-throughput assay for small molecule destabilizers of the KRAS oncoprotein. PLOS ONE 9:e103836.
Chang W, Gao X, Han Y, Du Y, Liu Q, Wang L, Tan X, Zhang Q, Liu Y, Zhu Y, Yu Y, Fan X, Zhang H, Zhou W, Wang J, Fu C, Cao G. 2014. Gene expression profiling-derived immunohistochemistry signature with high prognostic value in colorectal carcinoma. Gut 63:1457-1467.
Choi H M T, Chang J Y, Trinh L A, Padilla J E, Fraser S E, Pierce N A. 2010. Programmable in situ amplification for multiplexed imaging of mrna expression. Nature Biotechnology 28:1208-1212.
Coleman M L, Sahai E A, Yeo M, Bosch M, Dewar A, Olson M F. 2001. Membrane blebbing during apoptosis results from caspase-mediated activation of ROCK I. Nature Cell Biology 3:339-345.
De Simone A, Evanitsky M N, Hayden L, Cox B D, Wang J, Tornini V A, Ou J, Chao A, Poss K D, Di Talia S. 2021. Control of osteoblast regeneration by a train of erk activity waves. Nature 590:129-133.
Di Micco R, Fumagalli M, Cicalese A, Piccinin S, Gasparini P, Luise C, Schurra C, Garre' M, Nuciforo P G, Bensimon A, Maestro R, Pelicci P G, d'Adda di Fagagna F. 2006. Oncogene-induced senescence is a DNA damage response triggered by DNA hyper-replication. Nature 444:638-642.
Frost J A, Geppert T D, Cobb M H, Feramisco J R. 1994. A requirement for extracellular signal-regulated kinase (ERK) function in the activation of AP-1 by ha-ras, phorbol 12-myristate 13-acetate, and serum. PNAS 91:3844-3848.
Fuerer C, Nusse R. 2010. Lentiviral vectors to probe and manipulate the wnt signaling pathway. PLOS ONE 5:e9370.
Gagliardi P A, Dobrzyński M, Jacques M A, Dessauges C, Ender P, Blum Y, Hughes R M, Cohen A R, Pertz O. 2020. Collective ERK/Akt Activity Waves Orchestrate Epithelial Homeostasis by Driving Apoptosis-Induced Survival. bioRxiv.
Gjorevski N, Nikolaev M, Brown T E, Mitrofanova O, Brandenberg N, DelRio F W, Yavitt F M, Liberali P, Anseth K S, Lutolf M P. 2022. Tissue geometry drives deterministic organoid patterning. Science 375:eaaw9021.
Haigis K M, Kendall K R, Wang Y, Cheung A, Haigis M C, Glickman J N, Niwa-Kawakita M, Sweet-Cordero A, Sebolt-Leopold J, Shannon K M, Settleman J, Giovannini M, Jacks T. 2008. Differential effects of oncogenic K-ras and N-ras on proliferation, differentiation and tumor progression in the colon. Nature Genetics 40:600-608.
Hino N, Rossetti L, Marin-Llaurado A, Aoki K, Trepat X, Matsuda M, Hirashima T. 2020. ERK-mediated mechanochemical waves direct collective cell polarization. Developmental Cell 53:646-660.
Hiratsuka T, Fujita Y, Naoki H, Aoki K, Kamioka Y, Matsuda M. 2015. Intercellular propagation of extracellular signal-regulated kinase activation revealed by in vivo imaging of mouse skin. eLife 4:e05178.
Ishii M, Tateya T, Matsuda M, Hirashima T. 2021. Retrograde ERK activation waves drive base-to-apex multicellular flow in murine cochlear duct morphogenesis. eLife 10:e61092.
Johnson H E, Toettcher J E. 2019. Signaling dynamics control cell fate in the early Drosophila embryo. Developmental Cell 48:361-370.
Kabiri Z, Greicius G, Zaribafzadeh H, Hemmerich A, Counter C M, Virshup D M. 2018. Wnt signaling suppresses MAPK-driven proliferation of intestinal stem cells. The Journal of Clinical Investigation 128:3806-3812.
Kim K, Doi A, Wen B, Ng K, Zhao R, Cahan P, Kim J, Aryee M J, Ji H, Ehrlich LIR, Yabuuchi A, Takeuchi A, Cunniff K C, Hongguang H, Mckinney-Freeman S, Naveiras O, Yoon T J, Irizarry R A, Jung N, Seita J, et al. 2010. Epigenetic memory in induced pluripotent stem cells. Nature 467:285-290.
Kita-Matsuo H, Barcova M, Prigozhina N, Salomonis N, Wei K, Jacot J G, Nelson B, Spiering S, Haverslag R, Kim C, Talantova M, Bajpai R, Calzolari D, Terskikh A, McCulloch A D, Price J H, Conklin B R, Chen H S V, Mercola M. 2009. Lentiviral vectors and protocols for creation of stable hesc lines for fluorescent tracking and drug resistance selection of cardiomyocytes. PLOS ONE 4:e5046.
Le A P, Rupprecht J-F, Mège R-M, Toyama Y, Lim C T, Ladoux B. 2021. Adhesion-mediated heterogeneous actin organization governs apoptotic cell extrusion. Nature Communications 12:397.
Leach J D G, Vlahov N, Tsantoulis P, Ridgway R A, Flanagan D J, Gilroy K, Sphyris N, Vázquez E G, Vincent D F, Faller W J, Hodder M C, Raven A, Fey S, Najumudeen A K, Strathdee D, Nixon C, Hughes M, Clark W, Shaw R, S:CORT consortium, et al. 2021. Oncogenic BRAF, unrestrained by TGFβ-receptor signalling, drives right-sided colonic tumorigenesis. Nature Communications 12:3464.
Lee B-C, Lee J Y, Kim J, Yoo J M, Kang I, Kim J-J, Shin N, Kim D J, Choi S W, Kim D, Hong B H, Kang K-S. 2020. Graphene quantum dots as anti-inflammatory therapy for colitis. Science Advances 6:eaaz2630.
Li H, Liu S, Jin R, Xu H, Li Y, Chen Y, Zhao G. 2021. Pyrvinium pamoate regulates MGMT expression through suppressing the wnt/β-catenin signaling pathway to enhance the glioblastoma sensitivity to temozolomide. Cell Death Discovery 7:288. Marchiando A M, Shen L, Graham W V, Edelblum K L, Duckworth C A, Guan Y, Montrose M H, Turner J R, Watson A J M. 2011. The epithelial barrier is maintained by in vivo tight junction expansion during pathologic intestinal epithelial shedding. Gastroenterology 140:1208-1218.
Matsubayashi Y, Ebisuya M, Honjoh S, Nishida E. 2004. ERK activation propagates in epithelial cell sheets and regulates their migration during wound healing. Current Biology 14:731-735.
Mills J C, Stone N L, Erhardt J, Pittman R N. 1998. Apoptotic membrane blebbing is regulated by myosin light chain phosphorylation. The Journal of Cell Biology 140:627-636.
Miyoshi H, Stappenbeck T S. 2013. In vitro expansion and genetic modification of gastrointestinal stem cells in spheroid culture. Nature Protocols 8:2471-2482.
Miyoshi H, VanDussen K L, Malvin N P, Ryu S H, Wang Y, Sonnek N M, Lai C-W, Stappenbeck T S. 2017. Prostaglandin E2 promotes intestinal repair through an adaptive cellular response of the epithelium. The EMBO Journal 36:5-24.
Närhi K, Järvinen E, Birchmeier W, Taketo M M, Mikkola M L, Thesleff I. 2008. Sustained epithelial beta-catenin activity induces precocious hair development but disrupts hair follicle down-growth and hair shaft formation. Development 135:1019-1028.
Nguyen T N Q, Jeannesson P, Groh A, Guenot D, Gobinet C. 2015. Development of a hierarchical double application of crisp cluster validity indices: a proof-of-concept study for automated FTIR spectral histology. The Analyst 140:2439-2448.
Nusse Y M, Savage A K, Marangoni P, Rosendahl-Huber A K M, Landman T A, de Sauvage F J, Locksley R M, Klein O D. 2018. Parasitic helminths induce fetal-like reversion in the intestinal stem cell niche. Nature 559:109-113.
Pérez-González C, Ceada G, Greco F, Matejčić M, Gómez-González M, Castro N, Menendez A, Kale S, Krndija D, Clark A G, Gannavarapu V R, Álvarez-Varela A, Roca-Cusachs P, Batlle E, Vignjevic D M, Arroyo M, Trepat X. 2021. Mechanical compartmentalization of the intestinal organoid enables crypt folding and collective cell migration. Nature Cell Biology 23:745-757.
Pokrass M J, Ryan K A, Xin T, Pielstick B, Timp W, Greco V, Regot S. 2020. Cell-cycle-dependent ERK signaling dynamics direct fate specification in the mammalian preimplantation embryo. Developmental Cell 55:328-340.
Potten C S, Kellett M, Roberts S A, Rew D A, Wilson G D. 1992. Measurement of in vivo proliferation in human colorectal mucosa using bromodeoxyuridine. Gut 33:71-78.
Regot S, Hughey J J, Bajar B T, Carrasco S, Covert M W. 2014. High-sensitivity measurements of multiple kinase activities in live single cells. Cell 157:1724-1734.
Reischmann N, Andrieux G, Griffin R, Reinheckel T, Boerries M, Brummer T. 2020. BRAFV600E drives dedifferentiation in small intestinal and colonic organoids and cooperates with mutant p53 and apc loss in transformation. Oncogene 39:6053-6070.
Reyes J, Chen J Y, Stewart-Ornstein J, Karhohs K W, Mock C S, Lahav G. 2019. Fluctuations in p53 signaling allow escape from cell-cycle arrest. Molecular Cell 73:1306.
Riemer P, Sreekumar A, Reinke S, Rad R, Schäfer R, Sers C, Bläker H, Herrmann B G, Morkel M. 2015. Transgenic expression of oncogenic BRAF induces loss of stem cells in the mouse intestine, which is antagonized by B-catenin activity. Oncogene 34:3164-3175.
Rozhok A I, DeGregori J. 2015. Toward an evolutionary model of cancer: considering the mechanisms that govern the fate of somatic mutations. PNAS 112:8914-8921.
Sanman L E, Chen I W, Bieber J M, Thorne C A, Wu L F, Altschuler S J. 2020. Generation and quantitative imaging of enteroid monolayers. Methods in Molecular Biology 2171:99-113.
Santos S D M, Verveer P J, Bastiaens P I H. 2007. Growth factor-induced MAPK network topology shapes erk response determining PC-12 cell fate. Nature Cell Biology 9:324-330.
Seno H, Miyoshi H, Brown S L, Geske M J, Colonna M, Stappenbeck T S. 2009. Efficient colonic mucosal wound repair requires trem2 signaling. PNAS 106:256-261.
Serra D, Mayr U, Boni A, Lukonin I, Rempfler M, Challet Meylan L, Stadler M B, Strnad P, Papasaikas P, Vischi D, Waldt A, Roma G, Liberali P. 2019. Self-organization and symmetry breaking in intestinal organoid development. Nature 569:66-72.
Serrano M, Lin A W, McCurrach M E, Beach D, Lowe S W. 1997. Oncogenic ras provokes premature cell senescence associated with accumulation of p53 and p16ink4a. Cell 88:593-602.
Shih Y-W, Chien S-T, Chen P-S, Lee J-H, Wu S-H, Yin L-T. 2010. Alpha-mangostin suppresses phorbol 12-myristate 13-acetate-induced MMP-2/MMP-9 expressions via alphavbeta3 integrin/FAK/ERK and NF-kappab signaling pathway in human lung adenocarcinoma A549 cells. Cell Biochemistry and Biophysics 58:31-44.
Sugimoto S, Ohta Y, Fujii M, Matano M, Shimokawa M, Nanki K, Date S, Nishikori S, Nakazato Y, Nakamura T, Kanai T, Sato T. 2018. Reconstruction of the human colon epithelium in vivo. Cell Stem Cell 22:171-176.
Thorne C A, Hanson A J, Schneider J, Tahinci E, Orton D, Cselenyi C S, Jernigan K K, Meyers K C, Hang B I, Waterson A G, Kim K, Melancon B, Ghidu V P, Sulikowski G A, LaFleur B, Salic A, Lee L A, Miller D M, Lee E. 2010. Small-Molecule inhibition of Wnt signaling through activation of casein kinase 1a. Nature Chemical Biology 6:829-836.
Thorne C A, Chen I W, Sanman L E, Cobb M H, Wu L F, Altschuler S J. 2018. Enteroid monolayers reveal an autonomous WNT and BMP circuit controlling intestinal epithelial growth and organization. Developmental Cell 44:624-633.
Tripurani S K, Wang Y, Fan Y-X, Rahimi M, Wong L, Lee M-H, Starost M F, Rubin J S, Johnson G R. 2018. Suppression of wnt/β-catenin signaling by EGF receptor is required for hair follicle development. Molecular Biology of the Cell 29:2784-2799.
Valon L, Davidović A, Levillayer F, Chouly M, Cerqueira-Campos F, Levayer R. 2020. Robustness of Epithelial Sealing Is an Emerging Property of Local ERK Feedbacks Driven by Cell Elimination. bioRxiv.
Vogelstein B, Fearon E R, Hamilton S R, Kern S E, Preisinger A C, Leppert M, Nakamura Y, White R, Smits A M, Bos J L. 1988. Genetic alterations during colorectal-tumor development. The New England Journal of Medicine 319:525-532.
Wang Y, Chiang I-L, Ohara T E, Fujii S, Cheng J, Muegge B D, Ver Heul A, Han N D, Lu Q, Xiong S, Chen F, Lai C-W, Janova H, Wu R, Whitehurst C E, VanDussen K L, Liu T-C, Gordon J I, Sibley L D, Stappenbeck T S. 2019. Long-term culture captures injury-repair cycles of colonic stem cells. Cell 179:1144-1159.
Warzych E, Pawlak P, Lechniak D, Madeja Z E. 2020. WNT signalling supported by MEK/ERK inhibition is essential to maintain pluripotency in bovine preimplantation embryo. Developmental Biology 463:63-76.
Wei G, Gao N, Chen J, Fan L, Zeng Z, Gao G, Li L, Fang G, Hu K, Pang X, Fan H Y, Clevers H, Liu M, Zhang X, Li D. 2020. Erk and MAPK signaling is essential for intestinal development through wnt pathway modulation. Development 147:185678.
Yui S, Azzolin L, Maimets M, Pedersen M T, Fordham R P, Hansen S L, Larsen H L, Guiu J, Alves M R P, Rundsten C F, Johansen J V, Li Y, Madsen C D, Nakamura T, Watanabe M, Nielsen O H, Schweiger P J, Piccolo S, Jensen K B. 2018. YAP/TAZ-dependent reprogramming of colonic epithelium links ECM remodeling to tissue regeneration. Cell Stem Cell 22:35-49.
Zhao R, Michor F. 2013. Patterns of proliferative activity in the colonic crypt determine crypt stability and rates of somatic evolution. PLOS Computational Biology 9:e1003082.

We claim:

1. An organoid comprising a plurality of cells derived from a human, said organoid comprising at least two compartments: a first compartment and a second compartment, wherein the first compartment comprises at least 10% undifferentiated stem cells, and the second compartment comprises at least 60% differentiated cells and less than 0.1% undifferentiated stem cells.

2. The organoid of claim 1 wherein the plurality of cells is derived from normal colon cells, or from adenomatous polyp.

3. The organoid of claim 1 wherein the plurality of cells is of tumor origin and therefore lacks contact inhibition.

4. The organoid of claim 1 wherein the plurality of cells forms a monolayer on a tissue culture plate.

5. The organoid of claim 1 wherein Erk kinase activity is lower in the first compartment than Erk kinase activity in the second compartment.

6. A method for preparing an organoid, comprising
a) digesting a biopsy sample obtained from human intestine with collagenase to form a single cell suspension,
b) transferring single cells in the suspension from step (a) onto one or more wells of a multi-well plate, said one or more wells being coated with extracellular matrix, and
c) allowing single cells to grow and self-organize to form a 2D organoid monolayer.

7. The method of claim 6, wherein the biopsy sample is derived from normal colon cells, or from an adenomatous polyp.

8. The method of claim 6, wherein the biopsy sample is of tumor origin.

9. A method of screening for bioactive compounds, using the organoid of claim 1, comprising
a) providing an organoid comprising a plurality of cells;
b) staining the plurality of cells with a plurality of markers, wherein the plurality of markers binds to different components of the plurality of cells;
c) applying a bioactive compound to some but not all organoids;
d) capturing images of the organoids;
e) analyzing and comparing a plurality of features based on the images obtained in (d), said plurality of features being selected from the group consisting of cell type, morphology, proliferation, metabolism, cytoskeleton, cytotoxicity, and combination thereof; and
f) determining the functionality of the bioactive compound based on the analysis and comparison performed in step (e).

10. The method of claim 9, wherein the organoid is loaded onto a multi-well plate in step (a).

11. The method of claim 9, wherein changes in the plurality of features are analyzed at a single cell level.

12. The method of claim 9, wherein the bioactive compound is selected from the group consisting of proteins, nucleic acids, and small molecule chemicals.

13. The method of claim 9, wherein the bioactive compound is small interfering RNA (siRNA).

14. A cell line derived from a human adenomatous polyp, wherein said cell line is capable of forming the organoid of claim 1.

15. The cell line of claim 14, wherein said cell line is GiLA1 cell line (University of Arizona Tissue Bank number CORp86b).

16. A cell line derived from a human adenomatous polyp designated GiLA1 cell line (University of Arizona Tissue Bank number CORp86b).
